(19) United States
(12) Patent Application Publication
(10) Pub. No.: US 2008/0131330 A1
(43) Pub. Date: Jun. 5, 2008

(54) Ultra-Violet Batch Water Treatment and Small Item Sterilization System

(75) Inventors: Donald E. Lyon, Hanwell (CA); Steve Millikin, St. Andrews (CA)

Correspondence Address: HUSCH BLACKWELL SANDERS LLP, 720 OLIVE STREET, SUITE 2400, ST. LOUIS, MO, 63101, US

(73) Assignee: UV LIGHT SCIENCES GROUP, INC., Miramichi, New Brunswick (CA)

(21) Appl. No.: 11/814,553

(22) PCT Filed: Jan. 24, 2006
(86) PCT No.: PCT/CA06/00088

§ 371 Date: Jul. 23, 2007

Related U.S. Application Data
(60) Provisional application No. 60/645,735, filed on Jan. 24, 2005.

(51) Int. Cl. B01J 19/12; G05D 99/00

(52) U.S. Cl. 422/105; 422/186.3

(57) ABSTRACT

A portable Ultra Violet (UV) sterilizer includes a body into which liquid, or small objects, may be placed. A bulb in the interior of the body and may be activated such that the radiation from the bulb kills DNA-based organisms present in the liquid, or on the objects, and thereby sterilizes the liquid or objects. Through the use of a highly reflective coating on the interior of the body, a lower wattage UV bulb may be used than would be normally necessary.

### FIELD OF THE INVENTION

The present invention relates to sterilization using Ultra-Violet (UV) radiation and, more particularly, to a UV batch water treatment and small item sterilization system.

### BACKGROUND

Sterilization may be considered the killing of all DNA-based organisms present on the object before the introduction of the object into a sterilizer. Such DNA-based organisms may include, but are not limited to, those organisms that cause: Anthrax; Severe Acute Respiratory Syndrome (SARS); and Avian influenza (also known as “Bird Flu”). Many methods are known for sterilizing liquid, such as water, to make the liquid safe to drink, i.e., to remove harmful microorganisms. For instance, the liquid may be heated to a rolling boil for a set period of time, the liquid may be filtered by a filter having a predetermined absolute pore size and/or the liquid may be chemically treated by chlorination or iodination.

It is known to sterilize liquids using UV radiation in a pass-through system, wherein the liquid passes through a sterilization chamber where it is irradiated.

Additionally, it is known to use UV radiation to perform batch sterilization. An example of batch sterilization may be found in U.S. Pat. No. 5,900,212 to Maiden et al., which discloses a hand-held water purification system including a pen-light sized UV light that is enclosed in a quartz cover. To protect the user from UV radiation in the air, the water purification system includes circuitry that will not allow the light to be turned on until it is completely submerged in water. The system may require stirring of the water to ensure adequate exposure of any harmful microorganisms to the UV radiation.

Known methods for sterilizing liquids may have drawbacks in metrics such as speed, efficacy or portability. Similarly, with respect to sterilization of solids, although large scale UV sterilizers, such as autoclaves, are available, such large scale sterilizers are heavy, awkward and do not travel well. Accordingly, there is a continuing need for improved systems for sterilizing liquids

### SUMMARY

A portable UV batch sterilizer includes a body into which liquid, or small objects, may be placed. The body includes a closed sleeve of UV transmissive material. A bulb may be introduced into the interior of the body within the sleeve and may be powered on such that the radiation from the bulb kills DNA-based organisms present in the liquid, or on the objects, and sterilizes the liquid or objects.

According to an embodiment of the present invention, there is provided an ultra-violet sterilization system. The ultra-violet sterilization system includes an opaque body defining a sterilization chamber having an access aperture through which matter to be sterilized may be received, an Ultra Violet radiation transmissive sleeve, with an open end and a closed end, mounted in the body such that the closed end is in the sterilization chamber and the open end is open to an exterior of the body and a base including: a source of Ultra Violet radiation; and a source of electrical power for the source of Ultra Violet radiation. The body is releasably mounted to the base such that the source of Ultra Violet radiation extends into the sterilization chamber within the sleeve.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures which illustrate example embodiments of this invention:

FIG. 1 is front, elevation view of an exemplary portable UV sterilizer according to an embodiment of the present invention;

FIG. 2 is a sectional view of the exemplary portable UV sterilizer of FIG. 1;

FIG. 3 is a bottom plan view of the exemplary portable UV sterilizer of FIG. 1;

FIG. 4 is a front, left, top perspective view of a second exemplary portable UV sterilizer;

FIG. 5 is a front, left, top perspective, exploded view of the second exemplary portable UV sterilizer of FIG. 4;

FIG. 6 is a front, left, top perspective, exploded view of a base of the second exemplary portable UV sterilizer of FIG. 4; and

FIG. 7 is a sectional view of a funnel filter of the second exemplary portable UV sterilizer of FIG. 4.

### DETAILED DESCRIPTION

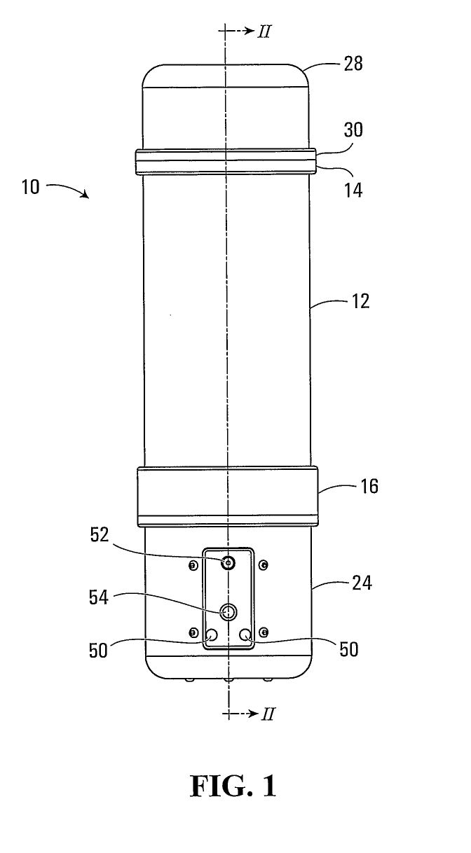

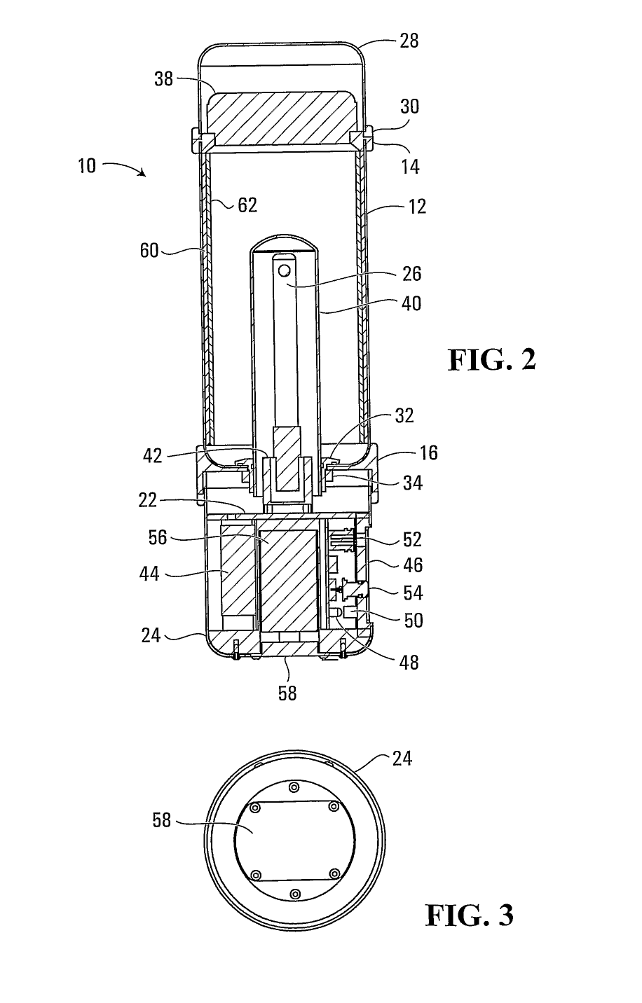

As illustrated in FIG. 1-3, a first exemplary embodiment of a portable UV sterilizer 10 generally includes a generally cylindrical body 12 with an open top and a bottom aperture, an electronics housing 24 releasably attached to the bottom of the body 12 and adapted to power and support a UV bulb 26 inside the body 20, and a stopper 38 for closing the top of the body 12.

Starting at the top, the body 12 has a main body threaded ring 14 attached at the open top thereof. The main body threaded ring 14 defines an external threaded surface and a top aperture. The stopper 38 is press-fit into the top aperture of the main body threaded ring 14 to close the top of the body 12. Also provided is a cup 28 having a cup threaded ring 30 with threads on an interior surface which cooperate with the threads on the main body threaded ring 14 to allow for the temporary attachment and detachment of the cup 28 to the top of the body 12.

The body 12 is formed of an opaque material, such as stainless steel, and may be insulated if it is desired to mitigate heat transfer to or from the interior of the body 12. The insulation may be any known means for insulating bottles such as, for instance, by a vacuum.

The interior of the body 12, i.e., the sterilization chamber, may be provided with highly reflective walls to reflect the germicidal UVC light from the bulb 26 back into the sterilization chamber. The wattage of UVC bulb required to achieve a given level of UVC exposure in a sterilization chamber with highly reflective walls will generally be lower than the wattage of UVC bulb required to achieve a given level of UVC exposure in a sterilization chamber without highly reflective walls.

Highly reflective walls may be obtained with a reflective coating 60, for example polished aluminum, sintered flouropolymers (such as Spectralon™ by Labsphere North Sutton, N.H.), thin-foil sintered flouropolymers with an aluminum backing or barium sulfate paint on a suitable backing substrate.

Many of the exemplary reflective coatings 60 may require protection from the matter that will be placed into the sterilization chamber. To protect the reflective coating 60, a lining or wall covering 62 of highly UVC transmissive material, such as GE 214 glass, may be used. Where the body 12 is formed as a cylinder without insulation, as illustrated, the wall covering 62 is expected to take the form of a cylinder with an outside dimension mating the inside dimension of the body 12 with reflective coating 60.

Rather than coating the interior surface of the body 12, the coating 60 may, for example, be barium sulfate paint applied to the exterior surface of the wall covering 62. Where the exterior surface of the wall covering 62 is coated, the wall covering 62 may be spaced from the wall of the body 12.

The releasable attachment between the body 12 and the electronics housing 24 is facilitated by a bottom bulkhead 16, which has an aperture corresponding to the bottom aperture in the body 12. The attachment of the body 12 to the bottom bulkhead 16 is accomplished by way of a bottom spigot 32 formed as an externally threaded hollow cylinder with a flange at one end. The cylinder of the bottom spigot 32 passes through the bottom aperture of the body 12 with the flange remaining in the interior of the body 12. The cylinder also passes through the aperture in the bottom bulkhead 16. A spigot jam nut 34 with interior threads is adapted to receive the cylinder of the bottom spigot 32 and may be advanced along the cylinder toward the flange to sandwich a portion of the body 12 surrounding the aperture and a portion of the bottom bulkhead 16 between the spigot jam nut 34 and the flange on the bottom spigot 32.

A quartz sleeve 40, with a closed end and an open end, is inserted into the sterilization chamber through the cylinder of the bottom spigot 32 and held in place by the bottom spigot 32 such that the closed end is in the sterilization chamber and the open end is open to the exterior of the body 12. Where the portable UV sterilizer 10 is to be used to sterilize liquid, the body 12, the bottom spigot 32 and the quartz sleeve 40 form a seal such that liquid does not reach the electronics housing 24.

The electronics housing 24 may also be formed of stainless steel. In the illustrated embodiment the electronics housing 24 is formed as a cylinder with an open top to receive electronics for operation of the portable UV sterilizer 10 and an aperture in the bottom to receive a battery pack 56. In the exemplary embodiment being described, the battery pack 56 holds eight standard AA batteries. The batteries may be one time use batteries or rechargeable batteries.

The electronics are based around an electronics chassis 18. An electronics cover plate 22 is attached to the top of the electronics chassis 18 and a bulb base socket 42 is attached to the electronics cover plate 22. A C-band Ultra-Violet (UVC) bulb 26 is received and held in place by the bulb base socket 42. The bulb 26 may be, for example, a 7 Watt G23 bulb. Where the bulb 26 is a standard GPXW bulb, the bulb base socket 42 may be a GX23 connector. The use of a “U-bulb” for the bulb 26 may be shown to minimize bulb footprint and to maximize available arc-length and, hence, efficiency.

A ballast 44 for stabilizing the current flow in the bulb 26 is received in a cavity on the back side of the electronics chassis 18. A control circuit board 20 is received In a cavity on the front side of the electronics chassis 18. The position of the control board 20 corresponds to a rectangular aperture in the front side of the electronics housing 24 and the control board 20 is covered by an interface plate 46 allowing access to the control board 20.

The control board 20 includes an alternating current (AC) adapter input receptacle 52 and the interface plate 46 allows access to the receptacle 52. Additionally, the control board 20 includes an activation switch 54. Furthermore, the control board 20 may have one or more light emitting diodes (LEDs) 48. Light emitted from the LEDs 48 may be conveyed to the outside of the electronics housing 24 by way of a light pipe 50 mounted in the interface plate 46.

The electronics chassis 18 includes a further cavity for receiving the battery pack 56. Access to the cavity in which the battery pack 56 is received is provided by an aperture in the bottom of the electronics housing 24, which aperture may be covered by a battery cover 58.

Once the contents of the electronics housing 24 have been assembled, the housing 24 may be attached to the body 12 by engagement with the bottom bulkhead 16. In particular, the bulb 26 is inserted within the quartz sleeve 40 while the electronics housing 24 is surrounded, at the top end, by a downwardly-depending skirt portion of the bottom bulkhead 16.

In overview, a liquid, or an object, to be sterilized may be placed into the sterilization chamber through the aperture in the main body threaded ring 14 with the stopper 38 removed. The stopper 38 may then be press-fit into the aperture in the main body threaded ring 14 so that the sterilization chamber is enclosed during sterilization. Using the activation switch 54, the bulb 26 is switched on and the liquid, or the object, is bathed in UVC radiation, thereby killing DNA-based organisms present in the liquid, or on the object.

Feedback to the user may be provided by the LEDs 48, for example to indicate to the user that the bulb 26 has been powered on and to indicate that the bulb 26 is healthy.

The source of electrical power for the bulb 26 may be one of multiple sources. The bulb 26 may, for example, receive power from batteries in the battery pack 56. Alternatively, the bulb 26 may receive power from an AC adapter via the AC adapter input receptacle 52. Where the batteries held by the battery pack 56 are rechargeable, the AC adapter input receptacle 52 may be used to supply power necessary to recharge the batteries. Additionally, the person of ordinary skill in the art should recognize that the bulb 26 may be powered by direct AC power, power from solar panels, power from a hand- or foot-cranked generator and power from a wind generator among other power sources.

Once powered on, the control board 20 may maintain power to the bulb 26 for a predetermined duration for example using timing circuit (not shown). The duration of activation of the bulb 26 may depend upon how much, and what type, of pathogenic material is to be neutralized, the volume of liquid to be sterilized and power rating of the bulb and output from the power source. As such, an interface (not shown) may be provided on the interface plate 46 to allow a user to adjust the duration of UVC exposure according to anticipated requirements.

During operation of the bulb 26, a bulb monitor circuit (not shown) may measure voltage being provided by batteries in the battery pack 56 and may measure a draw of current by the circuitry of the control board 20 used to operate the bulb 26. The battery voltage may be assigned a nominal value so that the measured value may be compared to the nominal value to quickly diagnose a problem, i.e., it may be quickly determined when the batteries are not supplying enough voltage to operate the bulb 26 to properly sterilize the matter to be sterilized. When the absolute voltage difference, between the measured value and the nominal value, exceeds a voltage threshold (i.e., the measured voltage falls outside of a predetermined tolerable band), the bulb monitor may indicate the condition by raising an auditory or visual (e.g., green light, red light) operating condition alarm.

Alternatively or additionally, the current draw by the bulb 26 may be assigned a nominal value so that the measured current value may be compared to the nominal value to quickly diagnose a problem, i.e., it may be quickly determined when the bulb 26 has burned out. When the absolute current difference, between the measured current value and the nominal value, exceeds a current threshold, the bulb monitor may indicate the condition by raising an auditory or visual operating condition alarm.

Rather than comparing the absolute current difference or the absolute voltage difference to an associated threshold, the measured value may be compared to a lower threshold computed by subtracting a small delta value from the nominal value. If the measured value is lower than the lower threshold, an alarm may be raised. Additionally, the measured value may be compared to an upper threshold computed by adding another (or the same) small delta value to the nominal value. If the measured value is higher than the upper threshold, an alarm may be raised. This will facilitate ensuring operation within a predefined range. Similarly, if either the absolute current difference or the absolute voltage difference exceeds an associated threshold, the bulb monitor may immediately shut down the portable UV sterilizer 10 and indicate to the user that the sterilization operation has failed, perhaps by activating a given one of the LEDs 48 with a predetermined color.

Even while the values measured by the bulb monitor are close to nominal, the bulb monitor may present the user with auditory or visual (e.g., green light, red light) operating condition alarms. Further condition monitoring, in the form of text-based notification of operating status and alarms, can also be included for mission critical applications where risk associated with failure is considered extremely high.

The portable UV sterilizer 10 may also sterilize any objects that fit into the sterilization chamber. To facilitate such small object sterilization, a UV transparent rack or other holding device (not shown) may be provided to place in the body 12 for holding small objects during sterilization.

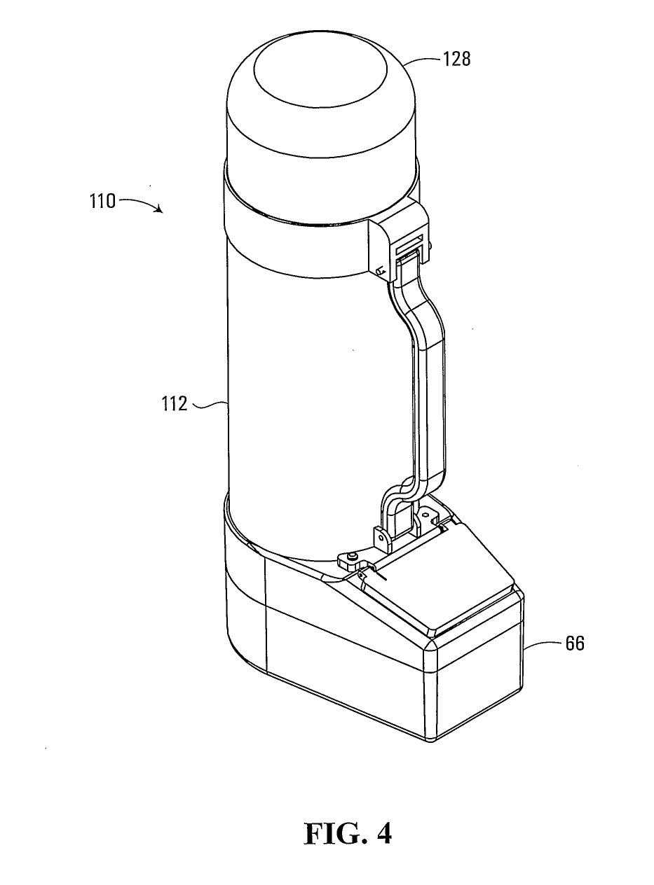

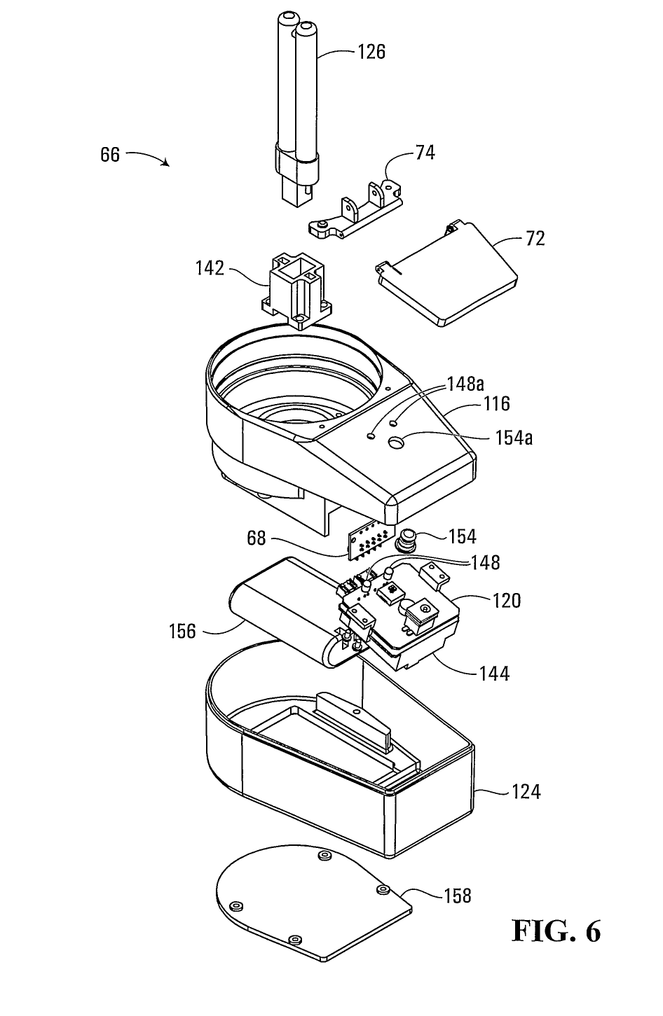

A second exemplary portable UV sterilizer 110 is illustrated in FIGS. 4-7 as having primary components that include a generally cylindrical body 112 and a base 66. The bottom of the body 112 is releasably received within the base 66 and the top of the body 112 is covered by a cup 128.

The body 112 is formed of an opaque material, such as stainless steel, and may be insulated if it is desired to mitigate heat transfer to or from the interior of the body 112. The insulation may be any known means for insulating bottles such as, for instance, by a vacuum.

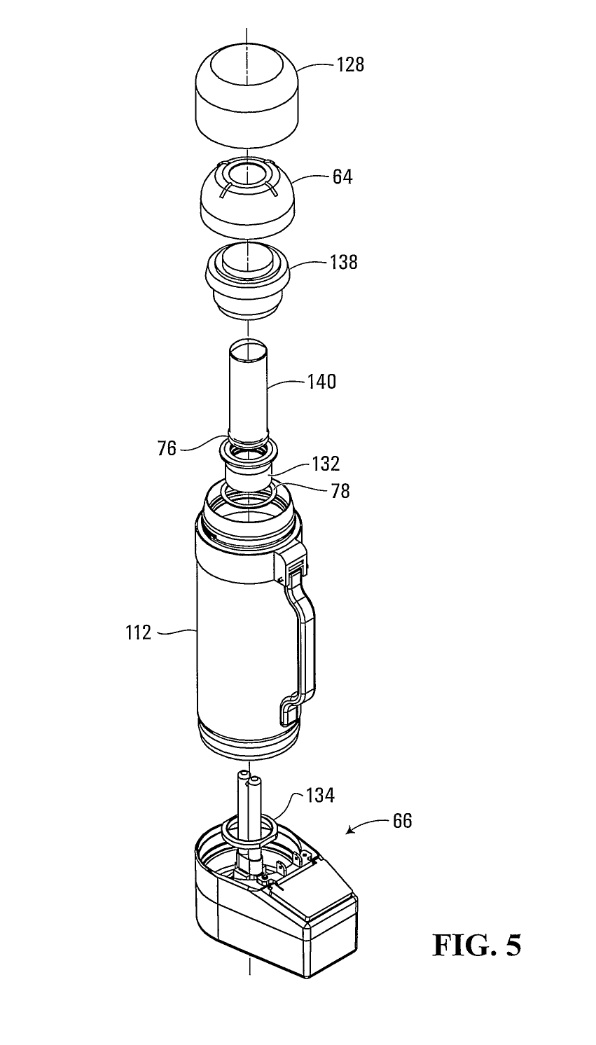

Starting at the top, the exploded view of FIG. 5 illustrates that top of the body 112 is releasably sealed by a stopper 138 that may be threaded into the open top of the body 112. A funnel 64 may fit over the stopper 138 and the cup 128 may be inverted and placed over funnel 64 and threaded onto the body 112.

The bottom aperture of the body 112 receives a bottom spigot 132. The bottom spigot 132 is formed as a hollow cylinder with a flange at one end, with the external surface of the cylinder being threaded. The cylinder of the bottom spigot 132 may be passed through a gasket 78 and the bottom aperture of the body 112, such that the flange remains in the interior of the body 112. A spigot jam nut 134 with interior threads may receive the cylinder of the bottom spigot 32 and may be advanced along the cylinder toward the flange to sandwich the gasket 78 and a portion of the body 112 surrounding the aperture between the spigot jam nut 134 and the flange on the bottom spigot 132.

A quartz sleeve 140, with a closed end and an open end, is inserted into the sterilization chamber through the cylinder portion of the bottom spigot 132 and held in place in the bottom spigot 132. The bottom of the quartz sleeve 140 may be surrounded with one or more silicone O-rings 76 to assist the formation of a seal between the quartz sleeve 140 and the bottom spigot 132.

The base 66 is formed of a bottom bulkhead 116 and an electronics housing 124. The bottom bulkhead 116 has an aperture corresponding to the bottom aperture in the body 112. The electronics housing 124, for enclosing the electronic for operation of the portable UV sterilizer 110, has an open bottom, for receiving a battery pack 156.

The electronics include a bulb base socket 142 attached to a control board 120. A C-band Ultra-Violet (UVC) bulb 126 is received and held in place by the bulb base socket 142. The bulb 126 may be, for example, a 7 Watt G23 bulb. The control board 120 attaches to a battery connector board 68 to receive electrical power from the battery pack 156. A ballast 144 is attached to the back side of the control board 120 for stabilizing the current flow in the bulb 126.

The control board 120 includes an activation switch 154 to which access is allowed through an aperture 154A in the bottom bulkhead 116. Furthermore, the control board 120 may have one or more light emitting diodes (LEDs) 148. The LEDs 148 may pass through corresponding apertures 148A in the bottom bulkhead 116 so that the state of the LEDs 148 may be viewed by a user.

To diminish the probability that dust or moisture reaches the control board 120 through the apertures 148A, 154A in the bottom bulkhead 116, a cover 72 may be provided. The cover 72 may be arranged to pivot open and closed through a connection to a pivot plate 74 that is attached to the bottom bulkhead 116.

The bottom bulkhead 116 includes a cavity for receiving the battery pack 156. Access to the cavity in which the battery pack 156 is received is provided by an aperture in the bottom of the electronics housing 124, which aperture may be covered by a battery cover 158.

Once the contents of the electronics housing 124 have been assembled, the body 112 may inserted into the assembled electronics and held in place by the bottom bulkhead 116. In particular, the bulb 126 may be inserted within the quartz sleeve 140 while the body 112 is surrounded, at the bottom end, by a up-standing skirt portion of the bottom bulkhead 116.

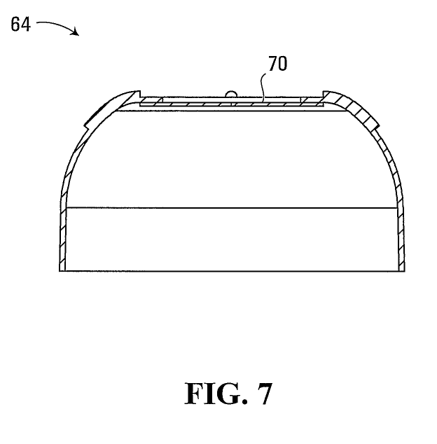

As illustrated in FIG. 7, the funnel 64 may have an aperture, passage through which is regulated by a filter 70. The filter 70 may be include a porous separator having a predetermined absolute pore size and may also include activated carbon or other standard liquid filtering materials.

In operation of the second exemplary portable UV sterilizer 110 of FIG. 4, the cup 128 and funnel 64 may be removed from respective attachment to the body 112 and the stopper 138 may then be removed from the aperture defined in the top of the body 112. The funnel 64 may be inverted and placed over the aperture in the top of the body 112. A liquid may then be introduced to the funnel 64 such that the liquid may flow through the filter 70 and into the body 112. The stopper 138 may then be re-threaded into the aperture. The filter 70 can be either be gravity fed, or power assisted. Power assistance can correspond to a manual pump or an AC or DC actuated pump (not shown).

The bulb 126 may then be powered on through use of the activation switch 154. Feedback to the user may be provided by the LEDs 148. First of all, to indicate to the user that the bulb 126 has been powered on and, second of all, to indicate that the bulb 126 is healthy.

It should be apparent to a person of ordinary skill in the art of sterilization that either bulb 26, 126 may emit pulsed white light radiation rather than UVC and still accomplish the sterilization task.

Whereas the portable UV sterilizers 10, 110, as described herein, are sized to be hand-held, and therefore portable, it should be clear that the disclosed UV sterilizer may be scaled to a larger size, in which case wheels may be required to move the larger scale UV sterilizer from place to place.

Advantageously, the UV sterilizer described above may be used to ensure clean drinking water for campers and personnel in the third world.

Furthermore, the use of a highly reflective coating allows use of a lower wattage UV bulb may be used than would be normally necessary. Accordingly, fewer batteries are necessary and the portable UV sterilizer is, therefore, beneficially lighter weight.

Other modifications will be apparent to those skilled in the art and, therefore, the invention is defined in the claims.

We claim:

1. An ultra-violet sterilization system comprising:
an opaque body defining a sterilization chamber having an access aperture through which matter to be sterilized may be received;
an Ultra Violet radiation transmissive sleeve, with an open end and a closed end, mounted in said body such that said closed end is in said sterilization chamber and said open end is open to an exterior of said body; and
a base including:
a source of Ultra Violet radiation; and
a source of electrical power for said source of Ultra Violet radiation;
said body being releasably mounted to said base such that said source of Ultra Violet radiation extends into said sterilization chamber within said sleeve.

2. The ultra-violet sterilization system of claim 1 wherein said Ultra Violet radiation is C-band Ultra Violet radiation.

3. The ultra-violet sterilization system of claim 1 further comprising a spigot for forming a seal between said body and said sleeve.

4. The ultra-violet sterilization system of claim 1 further comprising:
an Ultra Violet radiation transmissive lining within said sterilization chamber; and
a coating between said body and said lining to reflect said Ultra Violet radiation.

5. The ultra-violet sterilization system of claim 4 wherein said coating is polished aluminum.

6. The ultra-violet sterilization system of claim 4 wherein said coating is sintered flouropolymers.

7. The ultra-violet sterilization system of claim 4 wherein said coating is thin-foil sintered flouropolymers on an aluminum backing.

8. The ultra-violet sterilization system of claim 4 wherein said coating is barium sulfate paint on a backing substrate.

9. The ultra-violet sterilization system of claim 4 wherein said coating is barium sulfate paint on an external surface of said Ultra Violet radiation transmissive frame.

10. The ultra-violet sterilization system of claim 4 wherein said Ultra Violet radiation transmissive frame is formed of GE 214 quartz.

11. The ultra-violet sterilization system of claim 4 wherein said source of electrical power is direct current.

12. The ultra-violet sterilization system of claim 4 wherein said source of electrical power is alternating current.

13. The ultra-violet sterilization system of claim 1 further comprising a control circuit in communication with said source of Ultra Violet radiation and said a source of electrical power.

14. The ultra-violet sterilization system of claim 13 wherein said control circuit is adapted to determine a measure of voltage supplied by said source of electrical power.

15. The ultra-violet sterilization system of claim 14 wherein said control circuit is adapted to determine an absolute voltage difference between said measure of voltage and a predetermined voltage value.

16. The ultra-violet sterilization system of claim 15 wherein said control circuit is adapted to indicate that said absolute voltage difference exceeds a voltage difference threshold.

17. The ultra-violet sterilization system of claim 15 wherein said control circuit is adapted to interrupt supply of power to said source of Ultra Violet radiation responsive to said absolute voltage difference exceeding a voltage difference threshold.

18. The ultra-violet sterilization system of claim 14 wherein said control circuit is adapted to indicate that said measure of voltage exceeds an upper voltage threshold.

19. The ultra-violet sterilization system of claim 14 wherein said control circuit is adapted to indicate that a lower voltage threshold exceeds said measure of voltage.

20. The ultra-violet sterilization system of claim 13 wherein said control circuit is adapted to determine a measure of current drawn by said source of Ultra Violet radiation.

21. The ultra-violet sterilization system of claim 20 wherein said control circuit is adapted to determine an absolute current difference between said measure of current drawn and a predetermined current value.

22. The ultra-violet sterilization system of claim 21 wherein said control circuit is adapted to indicate that said absolute current difference exceeds a current difference threshold.

23. The ultra-violet sterilization system of claim 21 wherein said control circuit is adapted to interrupt supply of power to said source of Ultra Violet radiation responsive to said absolute current difference exceeding a current difference threshold.

24. The ultra-violet sterilization system of claim 20 wherein said control circuit is adapted to indicate that said measure of current drawn exceeds an upper current threshold.

25. The ultra-violet sterilization system of claim 20 wherein said control circuit is adapted to indicate that a lower current threshold exceeds said measure of current drawn.

26. The ultra-violet sterilization system of claim 13 wherein said control circuit is adapted to activate said source of Ultra Violet radiation for a time period, where a duration of said time period is selected by a user.

27. The ultra-violet sterilization system of claim 1 further comprising a funnel sized to fit over said access aperture, said funnel having a bottom aperture incorporating a filter.
